(19) United States
(12) Patent Application Publication
(10) Pub. No.: US 2025/0160786 A1
(43) Pub. Date: May 22, 2025

(54) INTRAVASCULAR ULTRASOUND IMAGING WITH CONTOUR GENERATION AND EDITING FOR CIRCULAR AND NON-CIRCULAR BLOOD VESSEL BORDERS

(71) Applicants: PHILIPS IMAGE GUIDED THERAPY CORPORATION, SAN DIEGO, CA (US); KONINKLIJKE PHILIPS N.V., EINDHOVEN (NL)

(72) Inventors: Vladimir ZAGRODSKY, EL DORADO HILLS, CA (US); Nikhil Sreedhar RAJGURU, SAN DIEGO, CA (US)

(21) Appl. No.: 18/841,748

(22) PCT Filed: Mar. 6, 2023
(86) PCT No.: PCT/EP2023/055523

§ 371 Date: Aug. 27, 2024

Related U.S. Application Data
(60) Provisional application No. 63/317,568, filed on Mar. 8, 2022.

(51) Int. Cl. A61B 8/12; A61B 8/00; G06T 7/60; G16H 30/40

(52) U.S. Cl. CPC A61B 8/12; A61B 8/463; A61B 8/465; A61B 8/5215; G06T 7/60; G16H 30/40; G06T 2207/10132; G06T 2207/20096; G06T 2207/20101; G06T 2207/20116; G06T 2207/30004

(57) ABSTRACT

A system includes a processor circuit that receives an intraluminal image of a body lumen. The processor circuit generates multiple anchors around an anatomical boundary. The anchors are generated based on a distance threshold representative of a distance between consecutive anchors and/or a curvature threshold representative of a curvature of the contour between consecutive anchors. The processor circuit generates a contour by connecting the multiple anchors and displays the intraluminal image with the contour.

### TECHNICAL FIELD

The present disclosure relates generally to generating and editing contours of vessel boundaries in intravascular images of peripheral vasculature. In particular, anchors are placed along a contour based on the shape of the contour to preserve the overall shape of the contour and promote intuitive and accurate manipulation.

### BACKGROUND

Intraluminal imaging is used in interventional treatment of peripheral vasculature as a diagnostic tool for assessing a diseased vessel, such as a peripheral vein, within the human body to determine the need for treatment, to guide the intervention, or to assess its effectiveness. An intraluminal imaging device including one or more ultrasound transducers is passed into the vessel and guided to the area to be imaged. The transducers emit ultrasonic energy. Ultrasonic waves are partially reflected by discontinuities in tissue structures (such as various layers of the vessel wall), red blood cells, and other features of interest. Echoes from the reflected waves are received by the transducer and passed along to an intraluminal imaging system. The imaging system processes the received ultrasound echoes to produce a cross-sectional intravascular (IVUS) image of the vessel where the device is placed.

To determine the location and extent of a diseased vessel, a physician generally makes measurements of the vessel based on the IVUS images received from the intraluminal imaging system. These measurements may help the physician to determine the blood flow at various locations along the vessel and include the diameter of the vessel and cross-sectional area of the vessel. An important step to making measurements of a vessel is to create a contour around the walls of the vessel as seen in an IVUS image. Various tools have been developed for this purpose. However, they generally assume that the vessel is circular and only allow contours to be generated around the center of the IVUS image. These limitations make creating and editing accurate contours difficult when peripheral vessels are imaged because these vessels are generally not circular. In addition, neighboring vessels which are not shown in the IVUS image center may need to be measured. Without the ability to create contours that accurately reflect the vessel shape, it is difficult for the physician to acquire accurate measurements of the vessel and correctly diagnose the location and extent of the issue and determine an appropriate remedy.

### SUMMARY

Embodiments of the present disclosure are systems, devices, and methods for generating and editing contours of vessel boundaries in intravascular images of peripheral vasculature. In particular, an intravascular imaging system may receive and display an IVUS image. The system may generate a contour outlining the boundary of the vessel shown in the IVUS image either automatically or in response to a user input. The system may then place multiple anchors in the form of dots around the contour. These anchors may be placed according to the shape of the contour. For example, the system may place an anchor along the contour if a minimum threshold distance to the nearest neighboring anchor is exceeded. If this distance is exceeded, the system may place an anchor when the local curvature of the contour exceeds a minimum threshold curvature, when the curvature of the contour changes from a positive curvature to a negative curvature or vice versa, or when a maximum distance to a neighboring anchor is met. After anchors are placed around the contour, the system may use spline interpolation to generate a path between all of the anchors creating a new contour. Placing anchors and interpolating between anchors advantageously allows generated contours to accurately reflect both circular and non-circular shapes and may be suited for both coronary and peripheral applications. In addition, the system can advantageously generate contours which cross a scan line of an IVUS image more than once and which are not required to be placed around the IVUS image center point. This allows for accurate measurement of neighboring vessels as well.

In an exemplary aspect of the present disclosure, a system is provided. The system includes a processor circuit configured for communication with an intraluminal imaging catheter and a display, wherein the processor circuit is configured to: receive an intraluminal image obtained by the intraluminal imaging catheter while the intraluminal imaging catheter is positioned within a body lumen; generate a plurality of anchors associated with an anatomical boundary of the body lumen; generate a contour corresponding to the anatomical boundary by connecting the plurality of anchors; and output, to the display, a screen display comprising the intraluminal image, the plurality of anchors within the intraluminal image, and the contour within the intraluminal image, wherein, to generate the plurality of anchors, the processor circuit is configured to determine a plurality of locations for the plurality of anchors based on: at least one distance threshold representative of a distance of the contour between the plurality of anchors; and at least one curvature threshold representative of a curvature of the contour between the plurality of anchors.

In some aspects, the processor circuit is configured to determine a location of a second anchor based on at least one of the distance or the curvature of the contour between a first anchor and the second anchor, and wherein the first anchor and the second anchor are consecutive. In some aspects, the at least one distance threshold comprises a minimum distance threshold, and wherein, to determine the location of the second anchor, the processor circuit is configured to place the second anchor only when the distance of the contour between the first anchor and the second anchor is equal to or greater in magnitude than the minimum threshold distance. In some aspects, the processor circuit is configured to change the minimum threshold distance. In some aspects, the at least one distance threshold comprises a maximum distance threshold, and wherein, to determine the location of the second anchor, the processor circuit is configured to always place the second anchor when the distance of the contour between the first anchor and the second anchor is equal to or greater in magnitude than the maximum threshold distance. In some aspects, the processor circuit is configured to change the maximum threshold distance. In some aspects, the at least one curvature threshold comprises a maximum negative curvature threshold, and wherein, to determine the location of the second anchor, the processor circuit is configured to place the second anchor when the curvature of the contour between the first anchor and the second anchor is equal to or less in magnitude than the maximum negative curvature threshold. In some aspects, the processor circuit is configured to place the second anchor only when the distance of the contour between the first anchor and the second anchor is equal to or greater in magnitude than the minimum threshold distance. In some aspects, the at least one curvature threshold comprises a maximum positive curvature threshold, and wherein, to determine the location of the second anchor, the processor circuit is configured to place the second anchor when the curvature of the contour between the first anchor and the second anchor is equal to or greater in magnitude than the maximum positive curvature threshold. In some aspects, the processor circuit is configured to place the second anchor only when the distance of the contour between the first anchor and the second anchor is equal to or greater in magnitude than the minimum threshold distance. In some aspects, to generate the contour, the processor circuit is configured to perform modified Akima piecewise cubic Hermite interpolation between the plurality of anchors. In some aspects, to generate the contour, the processor circuit is configured to perform interpolation between the plurality of anchors, wherein the processor circuit is configured to receive a user input to move a location of an anchor, wherein the processor circuit is configured to perform re-interpolation between only a subset of the plurality of anchors that is proximate to the anchor. In some aspects, the contour does not surround a position of the intraluminal imaging catheter in the intraluminal image. In some aspects, the system further includes the intraluminal imaging catheter.

In an exemplary aspect, a system is provided. The system includes a processor circuit configured for communication with an intraluminal imaging catheter and a display, wherein the processor circuit is configured to: receive an intraluminal image obtained by the intraluminal imaging catheter while the intraluminal imaging catheter is positioned within a body lumen; automatically generate a plurality of points associated with an anatomical boundary without receiving a user input identifying a plurality of locations for the plurality of points, wherein the plurality of points include a first point, a second point, and a third point; generate a contour corresponding to the anatomical boundary by connecting the plurality of points; and output, to the display, a screen display comprising the intraluminal image, the plurality of points within the intraluminal image, and the contour within the intraluminal image, wherein, to generate the plurality of points, the processor circuit is configured to determine the plurality of locations for the plurality of points such that a first distance of the contour between the first point and the second point is different than a second distance of the contour between the second point and the third point.

In an exemplary aspect, a system is provided. The system includes a processor circuit configured for communication with an intraluminal imaging catheter and a display, wherein the processor circuit is configured to: receive an intraluminal image obtained by the intraluminal imaging catheter while the intraluminal imaging catheter is positioned within a body lumen; generate a plurality of points associated with an anatomical boundary; generate a contour corresponding to the anatomical boundary by connecting the plurality of points; and output, to the display, a screen display comprising the intraluminal image, the plurality of points within the intraluminal image, and the contour within the intraluminal image, wherein, to generate the plurality of points, the processor circuit is configured to determine a plurality of locations for the plurality of points based on a change in a curvature of the contour between the plurality of points.

In an exemplary aspect of the present disclosure, a system is provided. The system includes an intravascular ultrasound (IVUS) imaging catheter; and a processor circuit configured for communication with the IVUS imaging catheter and a display, wherein the processor circuit configured to: receive an IVUS image obtained by the IVUS imaging catheter while the IVUS imaging catheter is positioned within a blood vessel; generate a plurality of anchors associated with an anatomical boundary; generate a contour corresponding to the anatomical boundary by performing interpolation to connect the plurality of anchors; and output, to the display, a screen display comprising: the IVUS image, the plurality of anchors within the IVUS image, and the contour within the IVUS image, wherein, to generate the plurality of anchors, the processor circuit is configured to determine a plurality of locations for the plurality of anchors based on: at least one distance threshold representative of a distance of the contour between two consecutive anchors, wherein the at least one distance threshold comprises a minimum distance threshold and a maximum distance threshold; and at least one curvature threshold representative of a curvature of the contour between the two consecutive anchors, wherein the at least one curvature threshold comprises a maximum negative curvature threshold and a maximum positive curvature threshold.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging system, according to aspects of the present disclosure.

FIG. 2 is a schematic diagram of a processor circuit, according to aspects of the present disclosure.

FIG. 3 is a diagrammatic view of a graphical user interface displaying a contour identifying the walls of a peripheral vessel, according to aspects of the present disclosure.

FIG. 4 is a diagrammatic view of a graphical user interface displaying a contour with automatically placed anchors identifying the walls of a peripheral vessel, according to aspects of the present disclosure.

FIG. 5 is a diagrammatic view of a section of a contour identifying a vessel wall, according to aspects of the present disclosure.

FIG. 6 is a diagrammatic view of a plot displaying the distance from one anchor the local curvature of a section of a contour, according to aspects of the present disclosure.

FIG. 7 is a diagrammatic view of a section of a contour identifying a vessel wall, according to aspects of the present disclosure.

FIG. 8 is a diagrammatic view of a plot displaying the distance from one anchor the local curvature of a section of a contour, according to aspects of the present disclosure.

FIG. 9 is a diagrammatic view of a section of a contour identifying a vessel wall, according to aspects of the present disclosure.

FIG. 10 is a diagrammatic view of a section of a contour identifying a vessel wall, according to aspects of the present disclosure.

FIG. 11 is a diagrammatic view of a contour prior to modification of an anchor, according to aspects of the present disclosure.

FIG. 12 is a diagrammatic view of a contour after modification of an anchor, according to aspects of the present disclosure.

FIG. 13 is a diagrammatic view of a graphical user interface displaying an interpolated contour with automatically placed anchors identifying the walls of a vessel different than the vessel in which the intravascular imaging device is positioned, according to aspects of the present disclosure.

FIG. 14 is a flow diagram for a method of generating a contour, automatically generating and placing anchors along the contour, and interpolating between the anchor, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the focusing system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

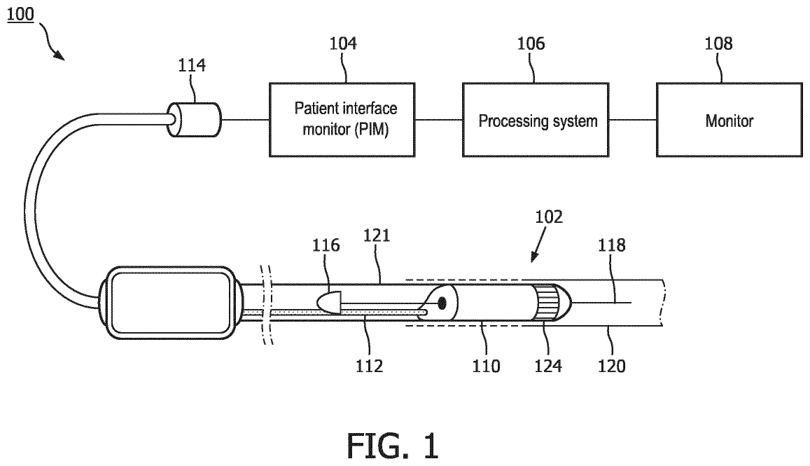

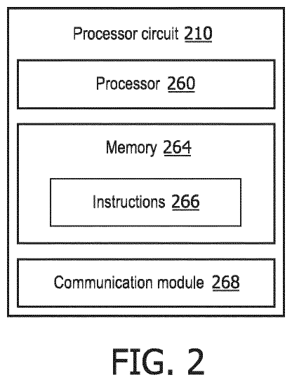

FIG. 1 is a diagrammatic schematic view of an intraluminal imaging system 100, according to aspects of the present disclosure. The system 100 may be a system comprising a processor circuit configured for communication with an intravascular imaging catheter 102 and a display 108. The system 100 may be a system comprising a processor circuit configured for communication with an intravascular ultrasound (IVUS) imaging catheter 102 and a display 108. The processor circuit may be the processor circuit 210 described with reference to FIG. 2. The display 108 may also be referred to as a monitor, as shown in FIG. 1. The intraluminal imaging system 100 can be an ultrasound imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, an processing system or console 106, and a monitor 108. The intraluminal imaging device 102 can be an ultrasound imaging device. In some instances, the device 102 can be an IVUS imaging device, such as a solid-state IVUS device. The intraluminal imaging device 102 may also be referred to as an intraluminal imaging catheter. The intraluminal imaging device may also be referred to as an intravascular ultrasound (IVUS) imaging catheter.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

The PIM 104 facilitates communication of signals between the IVUS console 106 and the scanner assembly 110 included in the IVUS device 102. This communication includes the steps of: (1) providing commands to integrated circuit controller chip(s) 206A and 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A and 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) 126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the transmission line bundle or cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

It is understood that the system 100 and/or device 102 can be configured to obtain any suitable intraluminal imaging data. In some embodiments, the device 102 may include an imaging component of any suitable imaging modality, such as optical coherence tomography (OCT), intracardiac echocardiography (ICE), etc. In some embodiments, the device 102 is an external ultrasound imaging device (e.g., an ultrasound probe, such as a transthoracic echocardiography probe) that obtains ultrasound imaging data while positioned outside of the patient body.

FIG. 2 is a schematic diagram of a processor circuit, according to aspects of the present disclosure. The processor circuit 210 may be implemented in the processing system 106 of FIG. 1. In an example, the processor circuit 210 may be in communication with the intraluminal imaging device 102, the x-ray imaging system 109, and/or the display 108 within the system 100. The processor circuit 210 may include a processor and/or communication interface. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the probe 110 and/or the processing system 106 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the probe 110, and/or the display or monitor 108. In that regard, the communication module 268 can be an input/output (I/O) device. In some instances, the communication module 268 facilitates direct or indirect communication between various elements of the processor circuit 210 and/or the probe 110 (FIG. 1) and/or the processing system 106 (FIG. 1).

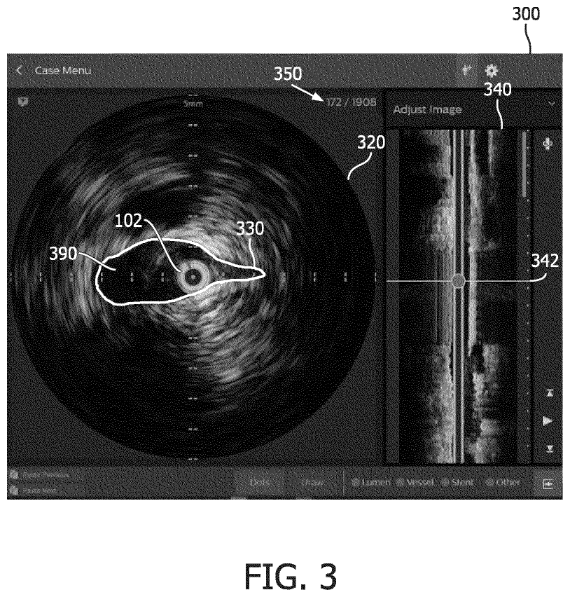

FIG. 3 is a diagrammatic view of a graphical user interface 300 displaying a contour 330 identifying the walls of a peripheral vessel, according to aspects of the present disclosure. The graphical user interface 300 may be displayed to the user of the system 100 during or after an imaging procedure. The graphical user interface includes an IVUS image 320 and an in-line digital or image longitudinal display (ILD) 340. In some embodiments, the graphical user interface 300, or any other interface described herein, may not include an ILD. In embodiments in which an ILD, such as the ILD 340, is displayed, it may advantageously provide a user of the system with a semi-three-dimensional view of the vessel in that a user may view a longitudinal view along the vessel as well data from radial cross-sectional views of IVUS images simultaneously. In this way, the ILD 340 may advantageously assist a user in navigating a three-dimensional anatomy of a patient. The processor circuit 210 (FIG. 2) may receive an intravascular image 320 obtained by the intravascular imaging catheter 102 (FIG. 1) while the intravascular imaging catheter 102 is positioned within a blood vessel. The processor circuit 210 (FIG. 2) may receive an IVUS image obtained by the IVUS imaging catheter while the IVUS imaging catheter is positioned within a blood vessel.

The graphical user interface 300 displays a contour overlaid on the IVUS image 320. The contour 330 may identify the inner walls defining the lumen of a peripheral blood vessel. For example, the inner region within the contour 330 or enclosed by the contour 330 may represent a cross-sectional shape or area through which blood may flow within the vessel imaged. In some embodiments, the contour 330 is generated automatically by a processing circuit (e.g., of the system 106), without a user input. In some embodiments, the contour 330 is generated in response to a user input. In some embodiments, the contour 330 may be generated in response to a user of the system 100 identifying an IVUS image 320 of interest. For example, during an imaging procedure, the system may acquire an IVUS image 320 of the IVUS images acquired by the intravascular device 102 (FIG. 1). The physician may identify the IVUS image 320 as displaying a region along the imaged vessel of particular interest. For example, the IVUS image 320 may correspond to a region of reduced blood flow along the vessel. Reduced blood flow may occur within the imaged vessel as a result of various conditions such as a constriction of the vessel caused by neighboring anatomies compressing the vessel, an obstruction within the vessel, such as a blood clot, a hardening of the walls of the vessel, or various other conditions. In addition, a region of interest may correspond to a lesion, a damaged or diseased section of the vessel. The region of interest may also correspond to a healthy region of the vessel. After the user of the system 100 has identified the IVUS image 320, the system 100 may generate the contour 330 identifying the cross-sectional shape of the vessel. While the contour 330 is a lumen border in the illustrated embodiment of FIG. 3, the contour 330 can be any anatomical or user drawn border or boundary of the vessel. For example, the contour 330 can be any suitable anatomical boundary of the vessel, such as the outer border of the vessel, the adventitia-media border, the intima-media border, internal elastic lamina (IEL) border, external elastic lamina (EEL) border, and/or other suitable boundary of the vessel.

The system 100 may generate the contour 330 in response to various user inputs. For example, and as shown in FIG. 3, the contour 330 may be generated based on a drawing by the user. The user may move a mouse such that the pointer of the mouse moves along the areas of the IVUS image at which the user wishes to create a contour. Alternatively, the user may move their finger or a stylus along a touch screen, use input buttons, such as arrow keys on a computer keyboard or any other buttons to identify the locations to correspond to the contour 330, or may input the shape of the contour 330 by another user input device. In that regard, the user input can trace out the vessel border and/or the lumen border. In an example in which a mouse is used, the user may depress a button on the mouse while moving the pointer along the desired contour 330 and release the button when the outline is completed. In some embodiments, the user input can be an input to initiate automatic generation of the contour 330 by the processor circuit. In that regard, the processor circuit can identify and output the contour 330 without user input, but a user input (e.g., selection of a find borders soft key or button) can be provided to cause the processor to perform the steps.

In another embodiment, the user may select one or more locations along the contour 330 to input to the system 100 the locations at which to generate the contour 330. In such an embodiment, the system 100 may generate a dot, or any other symbol or graphical representation, overlaid on the IVUS image 320 at each selected location. For example, the user may select one or more locations at which the inner lumen 390 of the vessel meets the outer wall. After all of the desired locations are selected, the user may input to the system to generate a contour based on the points selected. These points may be identified by clicking a mouse, as explained earlier, by touching the user's finger or stylus to a touch screen, by an input from a button, or by any other input device.

In some embodiments, the contour 330 may be automatically generated by the processor circuit (e.g., the processor circuit 210 of FIG. 2) without a user input. For example, the system 100 may use various image processing techniques to identify the inner lumen 390 and the walls of the vessel surrounding it. To do this, the system 100 may perform any suitable analysis of the received intraluminal image frames. For example, the system 100 may apply various image processing techniques such as edge identification, pixel-by-pixel analysis to determine transition between light pixels and dark pixels, filtering, or any other suitable techniques to identify relevant structures or locations within the received image frames. The system 100 may also employ various machine learning techniques. The system may generate a contour 330 based on this analysis and display the contour 330 to the user as shown in FIG. 3.

Examples of border detection, image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

In some embodiments, the system 100 may generate a contour 330 based on a previously generated contour. For example, during one imaging procedure, a contour may be generated either as directed by a user by drawing, by selecting locations and/or placing dots around the vessel wall within an IVUS image, or by automatic generation. This contour may be stored in the memory 264 (FIG. 2) of the system 100. In response to a user input to display the same IVUS image, the system 100 may retrieve or load the same contour and again generate the same contour that was previously created.

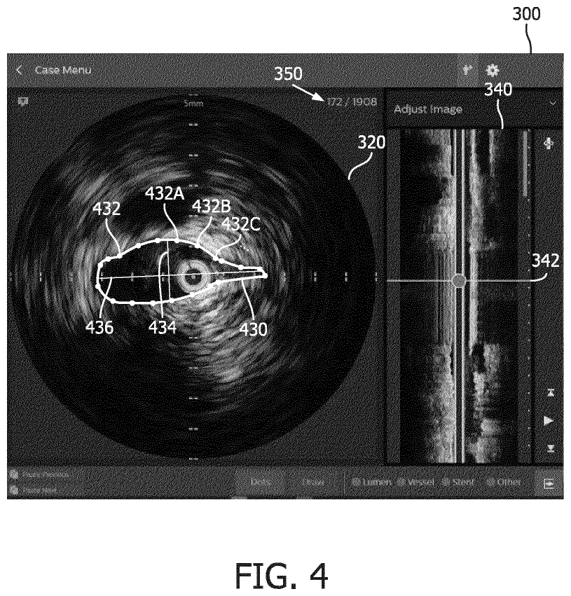

The generation of the contour 330 may assist the physician or user of the system 100 with performing measurements of the imaged vessel. For example, as will be discussed with more detail with reference to FIG. 4, measurements of the diameter, cross-sectional area, blood flow, or other metrics of the imaged vessel may be determined based on the contour 330 generated.

As shown in FIG. 3, the contour 330 may be of any suitable shape and need not be circular. As a result, the methods and systems described herein are equally suited to generated contours of cross-sectional vessel shapes in any location throughout a patient's body, including coronary vessels or peripheral vessels. In that regard, a coronary vessel may have a relatively more circular shape than a peripheral vessel. Indeed, some peripheral vessels have a much flatter cross-sectional profile.

The graphical user interface 300 also depicts the intravascular device 102. The device 102 may be seen within the imaged vessel but may identify structures, such as neighboring vessels or other anatomies not within the imaged vessel itself. In addition, as will be discussed hereafter, although the contour 330 is seen enclosing the intravascular device 102 (i.e. the contour 330 identifies the walls of the vessel through which the IVUS device 102 passes), the contour does not need to enclose the device 102. For example, the contour 330 may be placed at any suitable location within the IVUS image 320, including at locations which do not encompass the device 102.

The ILD 340 may assist a user of the system 100 to identify where along the imaged vessel the IVUS image 320 was obtained. The ILD 340 may be generated by the system 100 based on the IVUS images received by the device 102. The ILD 340 provides a longitudinal cross-sectional view of the blood vessel. In contrast, the IVUS image 320 is a tomographic or radial cross-sectional view of the blood vessel. The ILD 340 can be a stack of the intraluminal images acquired at various positions along the vessel, such that the longitudinal view of the ILD 440 is perpendicular to the radial cross-sectional view of the intraluminal image. In such an embodiment, the ILD 340 may show the length of the vessel, whereas an individual intraluminal image is a single radial cross-sectional image at a given location along the length. In another embodiment, the ILD 340 may be a stack of the intraluminal images acquired over time during the imaging procedure and the length of the ILD 340 may represent time or duration of the imaging procedure. The ILD 340 may be generated and displayed in real time or near real time during the pullback procedure. As each additional intraluminal image is acquired by the device 102 (FIG. 1), it may be added to the ILD 340. For example, at a point in time during the pullback procedure, the ILD 340 may be partially complete. In some embodiments, the processor circuit may generate an illustration of a longitudinal view of the vessel being imaged based on the received IVUS images. For example, rather than displaying actual vessel image data as the ILD 340 does, the illustration may be a stylized version of the vessel, with e.g., continuous lines showing the lumen border and vessel border. The ILD 340 may include an indicator 342 identifying the location along the ILD 340 at which the IVUS image 320 currently displayed was obtained.

The graphical user interface 300 may additionally include any other suitable intravascular imaging-related data, such as the indicator 350. The indicator 350, like the ILD 340, may convey which IVUS image is displayed in relation to all of the IVUS images obtained. For example, the indicator 350 may display a number indicating the order in which the IVUS image 320 was obtained in relation to all of the IVUS images obtained. The indicator 350, or any other indicators which may be included within the interface 300, may include other information, such as measurements relating to the IVUS image 320 or the contour 330, a name of a patient, the date of the procedure, conditions of the patient, annotations from caretakers, or any other information.

FIG. 4 is a diagrammatic view of a graphical user interface 300 displaying a contour 430 with automatically placed anchors 432 identifying the lumen wall of a peripheral vessel, according to aspects of the present disclosure. The processor circuit 210 may be configured to generate a plurality of anchors associated with an anatomical boundary of the blood vessel, as shown in FIG. 4 and described in more detail with reference to FIG. 4. The anchors 432 shown and described with reference to FIG. 4, as well as any other anchors shown and/or described throughout the present application may alternatively be referred to as anchor points, points, handles, dots, selectable graphical elements, indices, or any other similar term.

The contour 430 may be similar to the contour 330 of FIG. 3. The contour 430 may be based on the contour 330 in that the contour 430 is a result of smoothing the contour 330 and/or interpolating points along the contour 330. After the contour 330 of FIG. 3 is generated, the system 100 may perform a number of steps. These steps may include, among others, smoothing the lines of the contour 330, automatically generating and placing anchors 432 around the contour, and interpolating between the anchors 432 to create a new interpolated contour. In some embodiments, a smoothing step may be performed before an interpolation step. In other embodiments, a smoothing step may be performed after an interpolation step. In other embodiments, a smoothing step may be included as a part of an interpolation step. In one example, as a user of the system identifies the initial contour 330, the system 100 may identify multiple samples. These samples may include pixels within the image 320 defining the contour 330. In some embodiments, the processor circuit 210 may be configured to down sample the contour 330. Down sampling the contour 330 may include excluding one or more pixels of the pixels defining the contour 330. Down sampling the contour 330 may be included in a smoothing step or an interpolation step. In some embodiments, a smoothing step or an interpolation step may include interpolating a contour (e.g., the contour 330) at a lower resolution or a fixed resolution. In some embodiments, the curve 430 may be similarly down sampled as described or may be interpolated at a lower resolution. In some embodiments, the smoothing may use 2+1+2 anchors. For example, as a new segment is added, 2 anchors each from the ends of the curve and the new point may be added. Any or all of these methods of smoothing and/or interpolating may be applied to any steps described.

As described, the contour 330 and the contour 430 may not be completely identical, but they are both system-generated lumen borders. For example, the processor circuit can generate the contour 330 in one manner and the contour 430 in another manner, so that both the contour 330 and contour 430 are representative of the same anatomical border. All or a portion of the contour 330 and 430 may be identical. These steps may include additional steps before, after, or in between these steps. In some embodiments, one or more of these steps may be omitted, performed in a different order, or performed concurrently. These steps can be carried out by any suitable component within the system 100 and all steps need not be carried out by the same component.

After the contour 330 of FIG. 3 is completed, the system 100 may receive an input from the user. For example, this input may the release of a mouse button or other input button. The input may be removing a finger or stylus from contact with a touch screen. In other embodiments, a different button on an input device or within the graphical user interface 300 may selected to input that the contour 330 is completed. In some embodiments, the system 100 may assume that the contour 330 is a closed shape such that the first location of the contour 330 identified by the user must match the final location of the contour 330. In such an embodiment, the system 100 may generate a region of the contour 330 of the first identified point to the final identified point. In some embodiments, the contour 330 is a closed shape such that the first identified point and last identified point are connected.

Upon receipt of a user input indicating that the contour 330 is complete, the system may smooth the contour 330. The contour 430 shown in FIG. 4 may the result of the smoothing of the contour 330 of FIG. 3. The system 100 may smooth the lines of the contour 330 by any suitable means. For example, methods may include linear smoothing techniques, additive smoothing, filtering, moving averages, or any other suitable methods may be employed.

Upon receipt of a user input that the contour 330 is complete, the system 100 may also generate and place a number of automatically generated dots or anchors 432. The placement of the anchors 432 may based on the contour 330. In some embodiments, the locations of the anchors 432 can be directly on the contour 330 of FIG. 3. In some embodiments, the locations of the anchors 432 may not be directly on the contour 330, but are spaced from contour 330, such that the contour 430 overall corresponds to the contour 330 and anatomical border. In the anchors 432 are directly on the contour 430 shown in FIG. 4. The anchors 432 can be described as being placed around the contour 330 or the contour 430. The anchors 432 may be placed along the lines of the contour 430. The locations of the anchors 432 may be placed so as to preserve the overall shape of the contour. The locations of the anchors 432 may be placed according to an algorithm with a number of rules determining anchor placement.

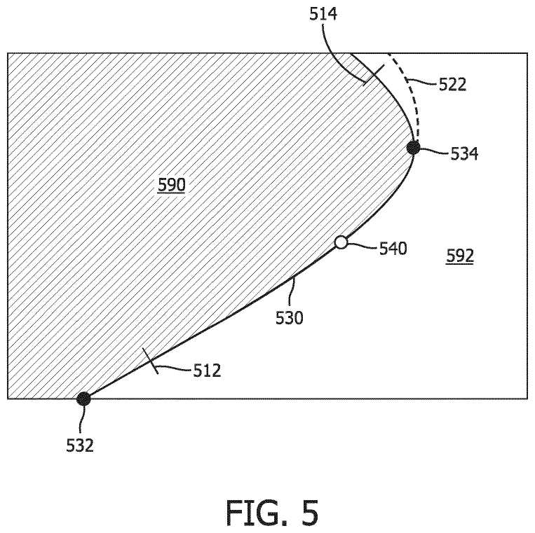

In brief, after a first anchor 432 is placed at a location along the contour 430, additional anchors 432 are placed depending on the distance along the contour 430 to the neighboring anchor 432 and the local curvature of the contour 430. The location of the first anchor 432 may correspond to a previous user input during the creation of the contour 330 (FIG. 3) or may correspond to a separate user input. In some embodiments, the first anchor 432 may be placed at a location corresponding to a first scanline in polar coordinates, at a location where the user first begins drawing a contour (e.g., the contour 330), or at an arbitrary location. For example, one anchor 432A may be placed at one location along the contour 430. Moving clockwise around the contour 430 from that first anchor 432A, the next anchor 432B may be placed at a location exceeding a minimum threshold distance from the first anchor 432A but not exceeding a maximum threshold distance. A minimum threshold distance between anchors ensures that anchors are not placed too close to one another, which may cause unwanted regions of the shape to be preserved or may make selecting an anchor 432 difficult for later editing. A maximum threshold distance ensures anchors are not spaced too far from one another ensuring that the overall shape of the lumen is preserved and making editing the contour more accurate and efficient. If both of these thresholds are met (i.e. for some location greater than the minimum threshold distance and less than the maximum threshold distance from the first anchor 432A), a location in which the curvature of the contour 430 exceeds a maximum positive curvature or drops below a minimum negative curvature may be selected as the location for an additional anchor 432B to be placed. These same rules may determine the location of the next anchor 432C placed and so on, until all anchors 432 have been placed at all appropriate locations around the contour 430 as shown in FIG. 4. Each of these rules will be described in more detail with reference to FIGS. 5-10. However, as shown in FIG. 4, these rules may dictate the automatic placement of the anchors 432 around a contour 430 (e.g., without a user input placing the anchors 432). In that regard, the processor circuit can automatically generate the anchors 432 and place the anchors 432 without receiving a user input identifying the locations for the anchors 432, the quantity of anchors 432, etc. In some embodiments, the processor circuit automatically generates the anchors 432 with no user input for all of the anchors 432. Some embodiments, the processor circuit automatically generates the anchors 432 with no user input for some of the anchors 432, a majority of the anchors 432, and/or all but one of the anchors 432. In some embodiments, the location of only a first anchor (a single anchor) may be related to a user input, but all of the other anchors 432 (the locations and/or the quantity) are automatically determined by the processor circuit without a user input.

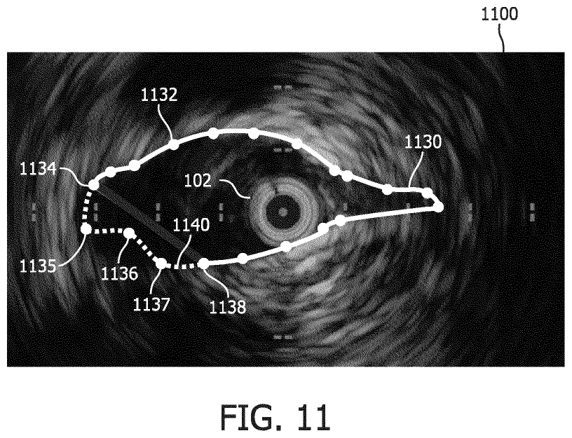

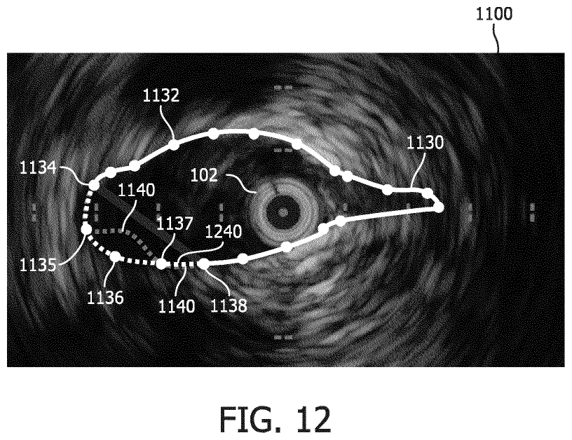

After all anchors 432 are placed around the contour 430 according to the algorithm described, the system 100 may interpolate between all the anchors 432 to further refine the shape of the contour 430. The processor circuit 210 (FIG. 2) may generate a contour corresponding to the anatomical boundary by connecting the plurality of anchors. The processor circuit 210 (FIG. 2) may generate a contour corresponding to the anatomical boundary by performing interpolation to connect the plurality of anchors. Similar to the smoothing process previously described, the interpolation may ensure that unintended movements of the hand, finger, or stylus during the creation of the contour 330 (FIG. 3) do not substantially impair the measurements of the vessel or the shape of the contour 430 in general. Various methods of interpolation may be used at this step. For example, spline-based interpolation algorithms, such as the modified Akima piecewise cubic Hermitian or Hermite interpolation (makima) may be employed. In other words, to generate the contour, the processor circuit 210 (FIG. 2) may be configured to perform modified Akima piecewise cubic Hermite interpolation between the plurality of anchors. The interpolation algorithm used may achieve parametric interpolation of 2D contours using the distance along the contour between anchors for synchronous interpolation in cartesian space and to deliver smooth, non-oscillating contours through sharp turns if anchors are placed in complex patterns or locations. The makima algorithm may be well suited to this purpose. In addition, the interpolation algorithm should ensure restricted impact of anchor manipulations on neighboring anchors as will be discussed in more detail with reference to FIGS. 11-12. The makima algorithm may be well suited to this purpose as well. Other interpolation algorithms may include inverse distance weighted algorithms, spline methods such as regularized or tension spline algorithms, kriging algorithms, radial basis function interpolation algorithms, piecewise cubic Hermitian interpolation, or any other suitable interpolation algorithms. In some embodiments, interpolation algorithms used by the present invention may include features similar to those described in the publication entitled “A New Method of Interpolation and Smooth Curve Fitting Based on Local Procedures,” by Hiroshi Akima, Journal of the ACM (JACM) 17.4, 1970, and/or the publication entitled “A Method of Bivariate Interpolation and Smooth Surface Fitting Based on Local Procedures,” by Hiroshi Akima, Communications of the ACM, 17.1, 1974, both of which are hereby incorporated by reference in their entirety.

As shown in FIG. 4, after the contour 430 is interpolated between the anchors 432, various measurements or metrics may be determined based on the contour 430. For example, diameters like the diameter 434 and the diameter 436 may be calculated. In some embodiments, the diameters 434 and 436 may be perpendicular to one another such that the system 100 may determine the distance between the inner walls of the vessel in one direction and another direction perpendicular to the first. In some embodiments, the diameters 434 and 436 can be chose to be the maximum diameters that are perpendicular to one another. In other embodiments, the diameters 434 and 436 are not perpendicular to one another. The lines illustrating the diameters 434 and 436 may both pass through a determined center point of the cross-sectional shape of the vessel or may not. The diameters 434 and/or 436 may be used to estimate a cross-sectional area of the vessel. The diameters 434 and/or 436 may also help to determine an estimated blood flow through the region shown in the IVUS image 320 or various other measurements.

The method described herein may be performed by the processor circuit 210 (FIG. 2) and may enable a number of advantages to a physician during an imaging procedure or while determining measurements of an imaged vessel. As noted, the described methods allow the system 100 to generate contours that accurately identify, reflect, or correspond to vessels of non-circular cross-sectional shapes, such as those of the peripheral vasculature. This allows for physicians to make more accurate measurements of imaged peripheral vessels. Physicians may subsequently be able to more accurately determine the location and extent of ailments of the imaged vessel and determine an appropriate remedy. In addition, the methods described herein allow the system to generate contours that cross a single scan line of an IVUS image multiple times, as opposed to only once. In the scan-converted images shown in FIGS. 3 and 4, each scan line extends radially between the center of the image to the outer periphery of the circumferential image frame; the image frame includes many scan lines around the circumference. This significantly broadens the possible vessel shapes around which a contour may be generated. In addition, the methods allow a system to generate a contour that is not around the center of the IVUS image or around the intravascular imaging device shown in the IVUS image. This advantageously allows physicians to make accurate measurements of neighboring vessels, or any other anatomy within or outside the imaged vessel of interest. In addition, the methods of the present disclosure allow more intuitive modification of contours by moving anchors and limiting the effects of reinterpolation locally to only neighboring anchors as will be described with more detail with reference to FIGS. 11-12. Additional aspects of automatically placing anchors will be described with reference to FIGS. 5-10 below. After the processor circuit 210 (FIG. 2) receives an intravascular image, generates a plurality of anchors associated with an anatomical boundary of the blood vessel, and generates a contour corresponding to the anatomical boundary by connecting the plurality of anchors, as described herein, the processor circuit 210 (FIG. 2) may output, to the display (e.g. the monitor 108 of FIG. 1), a screen display (e.g. the graphical user interface 300) comprising the intravascular image 320, the plurality of anchors 432 within the intravascular image 320, and the contours 430 within the intravascular image. The processor circuit 210 (FIG. 2) may output, to the display, a screen display comprising: the IVUS image, the plurality of anchors within the IVUS image, and the contours within the IVUS image.

FIGS. 5-10 describe how the processor circuit 210 (FIG. 2) may be configured to generate the plurality of anchors. To generate the plurality of anchors 432 (FIG. 4), the processor circuit 210 (FIG. 2) may be configured to determine a plurality of locations for the plurality of anchors 432 based on: at least one distance threshold representative of a distance of the contour between the plurality of anchors; and at least one curvature threshold representative of a curvature of the contour between the plurality of anchors, as will be described in more detail with reference to FIGS. 5-10. To generate the plurality of anchors, the processor circuit 210 (FIG. 2) may be configured to determine a plurality of locations for the plurality of anchors based on: at least one distance threshold representative of a distance of the contour between two anchors, wherein the at least one distance threshold comprises a minimum distance threshold and a maximum distance threshold; and at least one curvature threshold representative of a curvature of the contour between the two anchors, wherein the at least one curvature threshold comprises a maximum negative curvature threshold and a maximum positive curvature threshold. In some embodiments, the two anchors may be consecutive anchors. In some embodiments, the two anchors may be non-consecutive (e.g., there are intervening anchors between the two anchors). In that regard, starting from one anchor (e.g., a first anchor), the processor circuit can place another anchor (e.g., a second anchor) based on a distance and/or a curvature of at least the portion of the contour that starts from that first contour and extends away from the first anchor around the vessel/lumen boundary (e.g., towards the second anchor). In some instances, this can be referred to as placing the second anchor based on the distance and/or the curvature of the contour between the first anchor and the second anchor. For example, processor circuit determines the location of the second anchor based on the distance and/or the curvature of the segment of the contour starting from the first anchor and extending in the direction away from the first anchor around the vessel/lumen boundary (e.g., towards the second anchor).

It is noted that the steps described with reference to FIG. 3 and FIG. 4 may be completed in any order and may be independent of one another. For example, the processor circuit may be configured to identify a contour (e.g., the contour 330 of FIG. 3 or the contour 430 of FIG. 4) before, after, or while simultaneously generating anchor points (e.g., the anchor points 432). In some embodiments, the processor circuit (e.g., the processor circuit 210 of FIG. 2) can perform any of these steps iteratively, partially, in an interleaved manner, or in any other way. For example, the processor circuit 210 may be configured to generate one or more anchors corresponding to a portion of a contour, then generate a portion of the contour associated with those one or more points, and then proceed to generate other anchors and/or portions of the contour. Similarly, the processor circuit 210 may be configured to generate a portion of the contour, then generate one or more anchors corresponding to that portion of the contour, and then proceed to generate portions of the contour and/or other anchors.

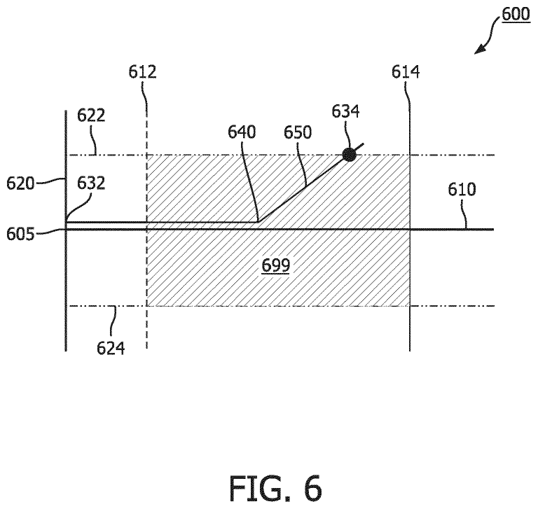

FIG. 5 is a diagrammatic view of a section of a contour 530 identifying a vessel and/or lumen wall, according to aspects of the present disclosure. FIG. 5 will be described with reference to FIG. 6 which is a diagrammatic view of a plot 600 displaying the distance from one anchor and the local curvature of a section of a contour, according to aspects of the present disclosure. Specifically, FIG. 5 illustrates a section of an exemplary contour 530 and FIG. 6 illustrates a plotted line 650 corresponding to the shape and behavior of the section of the contour 530 shown in FIG. 5. The algorithm determining the automated placement of anchors along a contour will be described with reference to both the contour 530 of FIG. 5 and the plot 600 of FIG. 6.

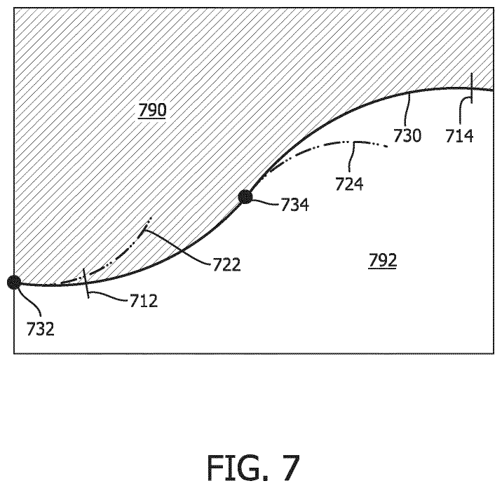

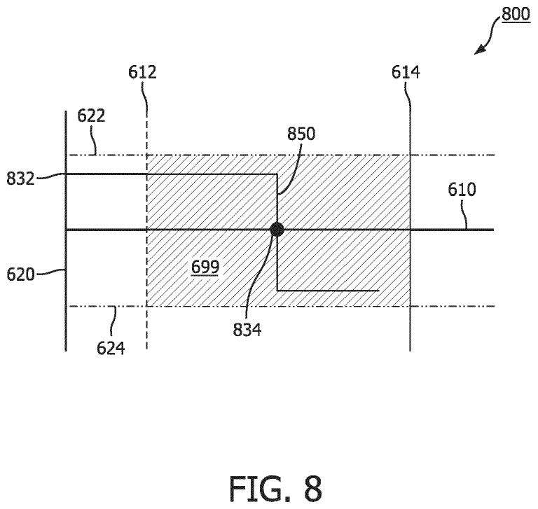

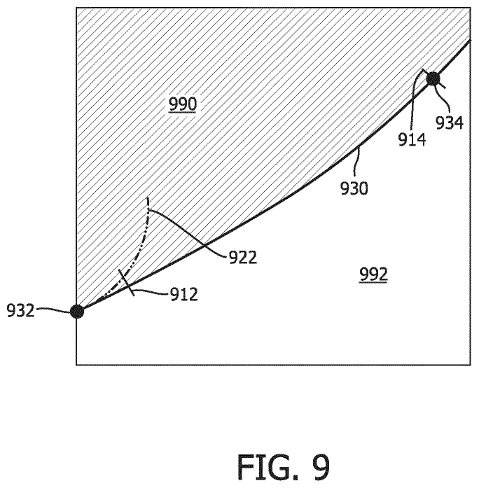

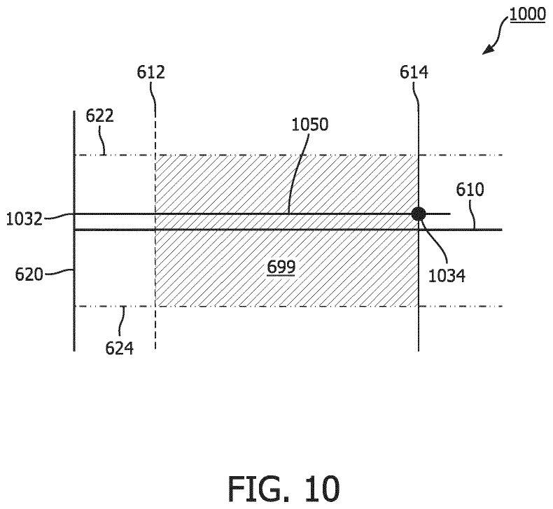

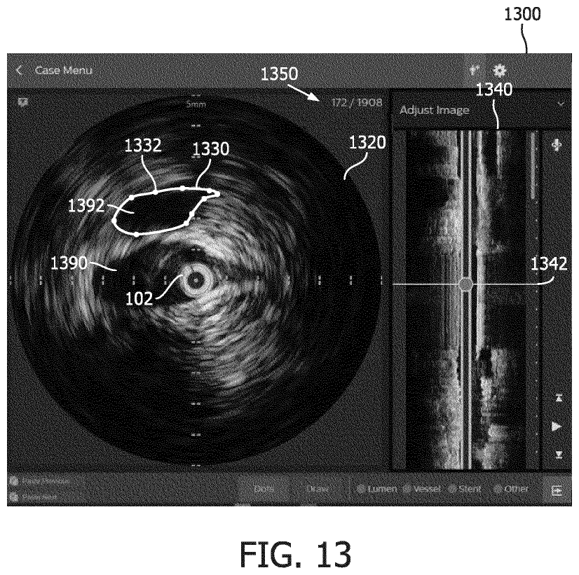

The contour 530 may be similar to the contours 330 and/or 430 described previously. For example, the contour 530 may be part of a closed shape identifying the wall of a vessel and/or lumen. As shown in FIG. 5, the contour 530 may identify a portion of a cross-sectional shape of an imaged vessel. The shaded region 590 may represent the inner lumen of the imaged vessel. In other words, blood may flow through the region 590 shown. The region 592 shown on the opposite side of the contour 530 may represent anatomy outside the lumen wall of the imaged vessel. Two anchor points, anchor 532 and anchor 534, are shown along the contour 530 in FIG. 5. In some embodiments, the anchor 532 may be referred to as a first anchor and the anchor 534 may be referred to as a second anchor. The anchor 532 and anchor 534 may be consecutive along the contour, adjacent along the contour, immediately following one another along the contour, next to one another along the contour. In other words, there may be no anchors between the anchor 532 and the anchor 534 or the anchor 532 and the anchor 534 (i.e. the first and second anchors) may be positioned along the contour without an intervening contour between them. Any of the anchors 432 of FIG. 4, the anchor 632 or the anchor 634 of FIG. 6, the anchor 732 or the anchor 734 of FIG. 7, the anchor 832 or the anchor 834 of FIG. 8, the anchor 932 or the anchor 934 of FIG. 9, the anchor 1032 or the anchor 1034 of FIG. 10, the anchors 1132, 1134, 1135, 1136, 1137, or 1138 of FIG. 11, or the anchors 1332 of FIG. 13 may be also be positioned in a similar consecutive manner as explained above. Additionally, a line 512 illustrates a minimum threshold distance, a line 514 illustrates a maximum threshold distance, line 522 illustrates a threshold curvature, and indicator 540 illustrates the location at which the curvature of the contour 530 begins to increase.

Referring now to FIG. 6, the plot 600 illustrates the shape and behavior of the section of the contour 530 shown in FIG. 5 in terms of distances between anchors and local curvatures of the contour 530. In FIG. 6, two axes are provided. The distance axis 610 extends horizontally from an origin 605. The distance axis represents the distance of any point along contour 530 from an anchor. In the example shown with reference to FIG. 5 specifically, the distance axis 610 illustrates the distance of any point from the anchor 532 (FIG. 5). The curvature axis 620 extends vertically in either direction from the origin 605 and illustrates the local curvature at any given point along the contour 530. The distance axis 610 crosses the curvature axis 620 at the origin 605, which is a point of zero curvature. The origin 605 may, therefore, correspond to a distance of zero from the anchor 532 and zero curvature. In other words, the origin 605 may represent the location of the anchor 532. In FIG. 6, the line 612, like the line 512 of FIG. 5, represents the minimum threshold distance.

The line 614, like the line 514 of FIG. 5, represents the maximum threshold distance. The line 622, like the line 522 of FIG. 5 represents the maximum positive threshold curvature, or the maximum curvature in a direction curving toward the region 590. The line 624 represents the maximum negative threshold curvature, or the maximum curvature in a direction curving toward the region 592. In this way, a positive curvature may refer to a curve in one direction while a negative curvature may refer to a curve in the opposite direction. In other words, throughout a straight section of the contour 530, the corresponding curvature would be zero. However, at a tight bend in the contour 530, the corresponding curvature would be either a positive or negative number depending on the direction of the bend. In some embodiments, the maximum negative threshold curvature may be referred to as a minimum threshold curvature. In addition, the maximum positive threshold curvature may be referred to as a maximum threshold curvature. Additional curvature thresholds may be implemented by the processor circuit 210 (FIG. 2) such as minimum positive curvature threshold or a minimum negative curvature threshold.

The minimum and maximum threshold distances 612 and 614 and the maximum positive and maximum negative threshold curvatures 622 and 624 together define a region 699. The region 699 is a rectangle or square, depending on the values of the minimum and maximum threshold distances 612 and 614 and the maximum and minimum threshold curvatures. The line 650 represents the contour 530 of FIG. 5. When a line corresponding to a contour (like the line 650 shown in FIG. 6) passes out of the region 699 by crossing either the maximum positive curvature 622, the maximum negative curvature 624, or the maximum distance 614 after first exceeding the minimum distance threshold 612, the system 100 will place an additional anchor point along the contour. Additionally, if a line corresponding to a contour either exceeds the maximum positive curvature 622 or is more negative than the maximum negative curvature 624 at the time the minimum distance threshold 612 is met, the system 100 will place an additional anchor point along the contour at the distance of the minimum threshold 612. In addition, if a line corresponding to a contour crosses the distance axis 610 at any point within the region 699, meaning the curvature goes from a positive to a negative value or vice versa, the system 100 will place an additional anchor point at that location. In this way, the processor circuit 210 (FIG. 2) may be configured to generate the plurality of anchors (e.g. anchors 432, anchor 532, or anchor 534) associated with an anatomical boundary of the blood vessel, wherein, to generate the plurality of anchors, the processor circuit is configured to determine a plurality of locations for the plurality of anchors based on: at least one distance threshold (e.g. the minimum distance threshold shown by the line 512 of FIG. 5 or the line 612 of FIG. 6, or the maximum distance threshold shown by the line 514 of FIG. 5 or the line 614 of FIG. 6) representative of a distance of the contour between the plurality of anchors, such as between the anchor 532 and 534 shown in FIG. 5 or between any of the anchors 432 shown in FIG. 4.

Turning to FIG. 5 as an example, the system may use these explained rules to determine the placement of the anchor point 534. Because the line 512 represents a minimum threshold distance from the anchor 532, the anchor 534 may not be placed at a location along the contour 530 between the anchor 532 and the line 512. This region is illustrated in plot 600 of FIG. 6 as the region between the vertical curvature axis 620 and the minimum threshold distance line 612. Regardless of the curvature of the contour at this region, an anchor point may not be placed. In this way, to determine the location of the second anchor (e.g. anchor 534 of FIG. 5 or any other anchor disclosed herein) the processor circuit 210 (FIG. 2) is configured to place the second anchor only when the distance of the contour between the first anchor and the second anchor is equal to or greater in magnitude than the minimum threshold distance (e.g. the minimum distance threshold shown by the line 512 of FIG. 5 or the line 612 of FIG. 6).

The exact minimum distance may depend on various factors. For example, the system 100 may determine the minimum threshold distance (i.e. the location of the line 512 of FIG. 5 and/or the location of the line 612 of FIG. 6) based on the field of view of the IVUS image 320 (FIG. 3) at the time, the type of intravascular device used, the type or size of the vessel imaged, or any other factors. For example, as a user of the system 100 zooms in within an IVUS image, the minimum threshold distance between anchors may decrease. In some embodiments, the user may be able to manually set the minimum threshold distance. The minimum threshold distance may be determined in terms of an actual distance along the cross-sectional shape of the vessel and may be in units of mm or any suitable metric of length. The minimum threshold distance may also be determined in terms of pixels of an IVUS image or display, an angle, or by any other metric. In this way, the processor circuit 210 (FIG. 1) may be configured to change, adjust, or modify the minimum threshold distance. The processor circuit 210 may be configured to change, adjust, or modify the minimum threshold distance in response to a user input or may do so automatically based on any of the parameters listed above.

As seen in FIG. 5, the curvature of the contour 530 near the anchor 532 may be quite small because the contour 530 is substantially straight at this location. This small positive curvature is observed within the plot 600 of FIG. 6. At the point 632 near the origin 605 representing the location of the anchor 532. In FIG. 5, this small positive curvature is maintained nearly constant, as shown by the region of the contour between the anchor 532 to the point 540, at which point the curvature begins to increase. This same behavior is observed in the plot 600 of FIG. 6. From the point 632 corresponding to the anchor 532, the curvature remains a relatively constant small positive value to the point 640 corresponding to the point 540 of FIG. 5 when the curvature begins to increase. As shown in FIG. 5, the curvature of the contour 530 remains below the maximum threshold curvature as illustrated by the line 522 until the point 534 at which the curvature of the contour 530 becomes greater than the threshold curvature 522. As the curvature of the contour 530 exceeds the maximum curvature 522, the anchor 534 is placed. Referring to FIG. 6, this behavior is observed as the line 650 increases showing the increase in curvature between the point 540 and 534 (FIG. 5). As the line 650 exceeds the maximum curvature line 622 and exits the region 699 at the point 634, the anchor 534 (FIG. 5) is placed. In this way, the processor circuit 210 (FIG. 2) may be configured to generate a plurality of anchors (e.g. anchors 532 or 534 of FIG. 5 or anchors 432 of FIG. 4) associated with an anatomical boundary of the blood vessel wherein, to generate the plurality of anchors, the processor circuit 210 is configured to determine a plurality of locations for the plurality of anchors based on at least one curvature threshold (e.g. the maximum positive curvature threshold shown by the line 622 of FIG. 6 or the line 522 of FIG. 5 or the maximum negative curvature threshold shown by the line 624 of FIG. 6) representative of a curvature of the contour between the plurality of anchors.

It is noted, that for purposes of this application, an additional anchor is placed when either the maximum positive curvature threshold or the maximum negative curvature is satisfied. A curvature value satisfies the maximum positive curvature threshold when it is on or above the line 622 and between the line 612 and the line 614. When the maximum positive curvature threshold is satisfied, an anchor consecutive to the anchor corresponding to the location 632 is placed if the minimum distance threshold has also been satisfied. A curvature value which satisfies the maximum positive curvature threshold can be equal to the maximum positive curvature threshold or may be greater in magnitude than the maximum positive curvature threshold.

Similarly, it is noted, that a curvature value satisfies the maximum negative curvature threshold when it is on or below the line 624 and between the line 612 and the line 614. When the maximum negative curvature threshold is satisfied, an anchor consecutive to the anchor corresponding to the location 632 is placed, if the minimum distance threshold has also been satisfied. A curvature value which satisfies the maximum negative curvature threshold can be equal to the maximum negative curvature threshold or may be less than, less in magnitude than, or, stated differently, more negative than the maximum negative curvature threshold.

It is also noted that values along the distance axis 610 and farther from the origin 605 are greater in value while values closer to the origin 605 are less in value. In this way, a distance value that is less than the minimum distance threshold 612, or positioned along to the left of the minimum distance threshold 612, or positioned closer to the origin 605 than the minimum distance threshold 612 do not satisfy, meet, or exceed the minimum distance threshold. Values equal to the minimum distance threshold 612, or the same distance from the origin 605 as the minimum distance threshold 612, as well as values greater than, or positioned to the right of, or positioned farther from the origin 605 than the minimum distance threshold 612 do satisfy, meet, and/or exceed the minimum distance threshold 612. The same terms may be used to describe values which satisfy, meet, or exceed the maximum distance threshold 614.

In this way, the at least one curvature threshold may comprise a maximum negative curvature threshold (e.g. shown by the line 624 of FIG. 6, and/or the line 724 of FIG. 7) and, to determine the location of the second anchor, the processor circuit 210 may be configured to place the second anchor when the curvature of the contour between the first anchor and the second anchor is equal to or less in magnitude than the maximum negative curvature threshold.

Similarly, the at least one curvature threshold may comprise a maximum positive curvature threshold (e.g. shown by the line 522 of FIG. 5, the line 622 of FIG. 6, the line 722 of FIG. 7, and/or the line 922 of FIG. 9) and, to determine the location of the second anchor, the processor circuit 210 is configured to place the second anchor when the curvature of the contour between the first anchor and the second anchor is equal to or greater in magnitude than the maximum positive curvature threshold.

FIG. 5 and FIG. 6 illustrate one exemplary way in which a processor circuit (e.g., the processor circuit 210 of FIG. 2) may be configured to automatically place anchor points along a contour within an IVUS image. Specifically, the processor circuit may be configured to place anchor points based on a curvature of the contour and a distance from a neighboring anchor point. However, in some embodiments, the processor circuit 210 may be configured to place anchor points along a contour based on only the curvature of the contour or only the distance from a neighboring anchor point.

For example, referring to FIG. 6, in an embodiment in which the processor circuit 210 automatically determines the placement of anchor points based on curvature but not based on distance, the plot 600 may not include the lines 612 or 614. In this way, the processor circuit may place an anchor point on the contour at any position in which the local curvature exceeds either the maximum positive threshold 622 or the maximum negative threshold 624.

In an embodiment in which the processor circuit 210 automatically determines the placement of anchor points based on a distance to the next neighboring anchor point, the plot 600 may not include the lines 622 or 624. In this way, the processor circuit may place an anchor point on the contour at any position at which the distance from a neighboring anchor point is equal to or exceeds the minimum distance threshold 612 but is equal to or less than the maximum distance threshold 614. In an embodiment in which the processor circuit 210 places anchor points along a contour based only on a distance measurement to a neighboring anchor point, the distance along the contour between neighboring anchor points may be equidistant or may not be equidistant. Specifically, the distance between neighboring anchor points along the contour which are placed according to distance and not curvature may be different. For example, as shown in FIG. 4, the anchors 432 may include the points 432A, 432B, and 432C. The points 432A, 432B, and 432C may represent different anchor points along the contour 430. The distance between the point 432A and the point 432B may be different than the distance between the point 432B and the point 432C even in an embodiment in which anchors are placed based on distance measurements and are not dependent on curvature.

FIG. 7 is a diagrammatic view of a section of a contour 730 identifying a vessel wall, according to aspects of the present disclosure. FIG. 7 will be described with reference to FIG. 8 which is a diagrammatic view of a plot 800 displaying the distance from one anchor and the local curvature of a section of a contour, according to aspects of the present disclosure. Specifically, like the relationship of FIGS. 5 and 6, FIG. 7 illustrates a section of an exemplary contour 730 and FIG. 8 illustrates a plotted line 850 corresponding to the shape and behavior of the section of the contour 730 shown in FIG. 7. Additional aspects of the algorithm for determining the automated placement of anchors along a contour will be described with reference to both the contour 730 of FIG. 7 and the plot 800 of FIG. 8.

The contour 730 shown in FIG. 7 may be similar to the contour 530. The contour 730 may be a part of a closed shape identifying the wall of a vessel and/or may identify a portion of a cross-sectional shape of an imaged vessel. The shaded region 790, like the region 590 of FIG. 5, may represent the inner lumen of the imaged vessel and the region 792 may represent anatomy outside the wall of the imaged vessel. Like FIG. 5, two anchor points, anchor 732 and anchor 734, are shown along the contour 730 in FIG. 7. Additionally, like lines 512 and 514 of FIG. 5, the line 712 illustrates a minimum threshold distance and line 714 illustrates a maximum threshold distance. Line 722 illustrates a maximum positive threshold curvature and line 724 represents a maximum negative threshold curvature.

Referring now to FIG. 8, the plot 800 may be similar to the plot 600. The plot 800 illustrates the shape and behavior of the section of the contour 730 shown in FIG. 7.

Because the line 712 represents a minimum threshold distance from the anchor 732, the anchor 734 may not be placed at a location along the contour 730 between the anchor 732 and the line 712. This region is illustrated in plot 800 of FIG. 8 as well.

Referring to FIG. 7, the curvature of the contour 730 may be largely uniform from the anchor 732 to the anchor 734. In addition, as shown compared to the exemplary curve 722 showing the maximum threshold curvature, the curvature between the anchor 732 and the anchor 734 is always less than then the threshold curvature shown by the line 722. Referring to FIG. 8, this same behavior is observed as the line 850 extends at a constant positive curvature from the location 832 near the anchor 732 to the location of the anchor 734 (FIG. 7) illustrated in FIG. 8 as the point 834. Because the curvature is less than the threshold curvature shown by the line 722 in FIG. 7 and the line 622 in FIG. 8 throughout this region, an additional anchor is not placed between the anchor 832 and the anchor 834.

However, referring to FIG. 7, at the location of the anchor 734, the curvature of the contour 730 is observed to immediately change from a positive curvature to a negative curvature. In addition, just as the curvature was a constant positive curvature from the anchor 732 to the anchor 734, the curvature is then a constant negative curvature throughout the remainder of the portion of the contour 730 displayed. This change in the curvature from a positive value to a negative value may be identified as an inflection point. An inflection point also occurs where the curvature changes from a negative value to a positive value. Referring to FIG. 8, an inflection point is observed to occur when the line 850 crosses the distance axis 610 as shown in FIG. 8. As a result of this inflection point, referring again to FIG. 7, the system 100 may place an additional anchor 734 at the inflection point. The point 834 shown in FIG. 8 corresponds to the anchor 734 of FIG. 7. In this way, to determine the location of the second anchor, the processor circuit 210 (FIG. 2) may be configured to place the second anchor when the curvature of the contour between the first anchor and the second anchor changes from positive to negative or from negative to positive. This change of the curvature from positive to negative or from negative to positive may also be described as a change of sign or a change of polarity.

In FIG. 7, it is noted that from the anchor 734 to the maximum threshold distance indicator 714, the constant negative curvature does not exceed the maximum negative threshold curvature. The maximum negative curvature is represented by the curve 724. Because the curvature of the contour 730 along the region between the anchor 734 and the maximum distance line 714 is less curved than the threshold curvature line 724, no anchor point is placed in this region. This is similarly shown in FIG. 8. The curvature as shown by the line 850 does not cross the lower threshold line 624 in FIG. 8. As a result, no additional anchor point is placed along this region.

As described with reference to FIGS. 5 and 6 previously, in some embodiments, the processor circuit may be configured to place anchor points on the contour according to either the curvature of the contour at a newly placed anchor point or the distance from a neighboring anchor point.

FIG. 9 is a diagrammatic view of a section of a contour 930 identifying a vessel wall, according to aspects of the present disclosure. FIG. 9 will be described with reference to FIG. 10 which is a diagrammatic view of a plot 1000 displaying the distance from one anchor and the local curvature of a section of a contour, according to aspects of the present disclosure. FIG. 9 illustrates a section of an exemplary contour 930 and FIG. 10 illustrates a plotted line 1050 corresponding to the shape and behavior of the section of the contour 930 shown in FIG. 9. Additional aspects of the algorithm for determining the automated placement of anchors along a contour will be described with reference to both the contour 930 of FIG. 9 and the plot 1000 of FIG. 10.

The contour 930 shown in FIG. 9 may be similar to the contour 530 and/or the contour 730. The contour 930 may be a part of a closed shape identifying the wall of a vessel and/or may identify a portion of a cross-sectional shape of an imaged vessel. The shaded region 990 may represent the inner lumen of the imaged vessel and the region 992 may represent anatomy outside the wall of the imaged vessel. Similarly, two anchor points, anchor 932 and anchor 934, are shown along the contour 930 in FIG. 9. Additionally, the line 912 illustrates a minimum threshold distance and line 914 illustrates a maximum threshold distance. Line 922 illustrates a maximum threshold curvature.

Referring now to FIG. 10, the plot 1000 may be similar to the plot 600 or the plot 800. The plot 1000 illustrates the shape and behavior of the section of the contour 930 shown in FIG. 9. Because the line 912 represents a minimum threshold distance from the anchor 932, the anchor 934 may not be placed at a location along the contour 930 between the anchor 932 and the line 912. This region is illustrated in plot 1000 of FIG. 10 as well.

Referring to FIG. 9, the curvature of the contour 930 is positive and largely uniform but well below the threshold curvature shown by the line 922 throughout the section shown. Referring to FIG. 10, this same behavior is observed as the line 1050 extends at a generally constant positive curvature from the location 1032 corresponding to the anchor 932 throughout the remainder of the plot 1000. Because the curvature is less than the threshold curvature shown by the line 922 in FIG. 9 and the line 622 in FIG. 10 throughout the entire region shown, an additional anchor is not placed anywhere between the anchor 832 and maximum threshold distance line 914 shown in FIG. 9 or the line 614 shown in FIG. 10. Because the curvature never exceeds the maximum curvature threshold line 622 shown in FIG. 10 and does not cross either the maximum negative curvature threshold hold line 624 or the distance axis 610, no anchor point is generated throughout this region, or throughout the region from the anchor point 932 and the maximum threshold line 914 shown in FIG. 9. As a result, an additional anchor point 934 is placed at the same location as the maximum distance threshold line 914 shown in FIG. 9. This is shown by the point 1034 in FIG. 10 at the same distance as the threshold line 614. In this way, the at least one distance threshold comprises a maximum distance threshold. In addition, to determine the location of the second anchor (e.g. the anchor 934 or any other anchor described herein), the processor circuit 210 (FIG. 2) may be configured to always place the second anchor when the distance of the contour between the first anchor and the second anchor is equal to or greater in magnitude than the maximum threshold distance.

The exact maximum distance may depend on various factors. For example, the system 100 may determine the maximum threshold distance (i.e. the location of the line 514 of FIG. 5 and/or the location of the line 614 of FIG. 6) based on the field of view of the IVUS image 320 (FIG. 3) at the time, the type of intravascular device used, the type or size of the vessel imaged, the local tortuosity of the contour, or any other factors. For example, as a user of the system 100 zooms in or out within an IVUS image, the maximum threshold distance between anchors may decrease or increase. In some embodiments, the user may be able to manually set the maximum threshold distance. The maximum threshold distance may be determined in terms of an actual distance along the cross-sectional shape of the vessel and may be in units of mm or any suitable metric of length. The maximum threshold distance may also be determined in terms of pixels of an IVUS image or display, an angle, or by any other metric. In this way, the processor circuit 210 (FIG. 1) may be configured to change, adjust, or modify the maximum threshold distance. The processor circuit 210 may be configured to change, adjust, or modify the maximum threshold distance in response to a user input or may do so automatically based on any of the parameters listed above.

The rules described with reference to FIGS. 5-10, specifically the implementation of a maximum and minimum distance thresholds and maximum positive and maximum negative curvature thresholds, advantageously allows the system 100 to automatically select the ideal number of anchor points around a contour, regardless of the shape of the contour. For example, regions of a high tortuosity of a non-circular contour may require many closely spaced anchor points to accurately outline the region. The rules described ensure that the region is properly outlined while still allowing for accurate and simple adjustment or manipulation by a user, as will be explained in more detail hereafter. In addition, the rules allow the system 100 to employ fewer anchor points for less tortuous regions of the contour, reducing unnecessary anchor points and improving usability. which allow for enough granularity in anchor placement that interpolation between the anchors results in a contour that follows a non-circular shape that can show up in the peripheral context. In this way, the system 100 may accurately outline an adjustable contour around cross-sectional lumens of any shape, including non-circular peripheral vessels. The processor circuit may be configured to determine a location of a second anchor based on at least one of the distance or the curvature of the contour between a first anchor and the second anchor, and wherein the first anchor and the second anchor are consecutive, as explained previously. The at least one distance threshold may comprise a minimum distance threshold (e.g. the minimum distance threshold shown by the line 512 of FIG. 5 and/or the line 612 of FIG. 6).

As described with reference to FIGS. 5-8 previously, in some embodiments, the processor circuit may be configured to place anchor points on the contour according to either the curvature of the contour at a newly placed anchor point or the distance from a neighboring anchor point.

FIG. 11 is a diagrammatic view of a contour 1130 prior to modification of an anchor 1136, according to aspects of the present disclosure. The image 1100 shown in FIG. 11 may be a portion or enlarged region of an IVUS image. The IVUS image may be a part of a graphical user interface similar to the graphical user interface 300 described with reference to FIGS. 3 and 4.

After a contour is created, smoothed, and/or interpolated, and anchors 1132 are positioned around the contour according to the algorithm described with reference to FIGS. 5-10, a user may wish to adjust the shape of the contour. As an example, a contour 1130 is shown in FIG. 11. The contour 1130 shown includes a region 1140 extending from the anchor 1134 to 1138. In the illustrated embodiment, this region 1140 has a different appearance from the rest of the contour 1130. For example, the line of the region 1140 may be of a different pattern as shown. In addition, it may be of any suitable color, weight or width, or of any suitable appearance. In some embodiments, the region 1140 may be of the same appearance as the rest of the contour 1130. The placement of anchors along a contour advantageously allows a user to more easily manually adjust the shape or position of the contour. The anchors provide multiple discrete places along the contour where the contour can be changed. In addition, by placing the contours according to the rules described with reference to FIGS. 5-10, the anchors are placed at places most likely to be adjusted, including regions of high tortuosity or inflection points. As described with reference to FIGS. 5-10, the processor circuit 210 (FIG. 2) may generate a plurality of anchors associated with an anatomical boundary of the blood vessel.

As shown in FIG. 11, the user may observe that one or more anchors, such as the anchor 1136, are not correctly placed (e.g., as a result of automatic contour generation and/or anchor placement by the processor circuit). In this situation, the user may wish to adjust the location of the anchor 1136 and thus cause the entire shape of the contour 1130 to more accurately conform with the shape of the lumen as observed in the image 1100. In this situation, a user may select the anchor 1136 and move it to any suitable location within the image 1100. The user may make this selection and movement of the anchor 1136 by any suitable means, including any input devices or methods previously described.

FIG. 12 is a diagrammatic view of a contour 1130 after modification of an anchor 1136, according to aspects of the present disclosure. As shown in FIG. 12, the anchor 1136 has been moved from its previous location within the image 1100 to a new position. After the anchor 1136 is moved, the system 100 may again interpolate the shape of the contour 1130 based on the location of the anchors. As a result, the lines extending from the moved anchor 1136 to the neighboring anchors 1135 and 1137 immediately adjacent to the anchor 1136 are not the only lines of the contour 1130 which are modified or interpolated again. However, the movement of the anchor 1136 does not cause a reinterpolation of the entire contour 1130 as shown. Rather, in the example shown in FIG. 12, only the lines extending from the anchor 1134 to the anchor 1138 as shown by the dotted line 1240 have been adjusted. For reference purposes, a depiction of the line 1140 from FIG. 11 is also shown radially inward of the line 1240. As shown in FIG. 12, the contour is changed from the middle anchor 1136 extending clockwise for a distance of two anchors, specifically anchors 1135 and 1134, as shown in the difference between the line 1140 before the change and the line 1240 after the change. However, there is no change to the placement of the line of the contour 1130 beyond the anchor 1134. The same limitation is observed in a counter-clockwise direction. The contour is changed from the middle anchor 1136 extending counter-clockwise for a distance of two anchors, specifically anchors 1137 and 1138, as shown in the difference between the line 1140 before the change and the line 1240 after the change. However, there is no change to the placement of the line of the contour 1130 beyond the anchor 1138. This limiting of reinterpolation for other anchors to only those local to the modified anchor advantageously allows for more accurate, fine-tuned adjustments to the contour 1130 ensuring that the contour reflects the cross-sectional shape of the vessel as accurately as possible.

In some embodiments, the extent to which the effect of modifying one anchor location is localized may be adjusted. For example, a user of the system or the system itself may determine, based on the vessel imaged, the type of catheter used, patient condition, personal preference, or any other relevant factors, that the effects of one anchor adjustment should be more or less limited. For example, after an anchor position is adjusted, the system 100 may limit reinterpolation to only the lines to the one neighboring anchor on either side of the moved anchor, instead of two. Similarly, the system 100 may allow for interpolation after an adjustment to the neighboring three anchors on either side, four, or more. The processor circuit 210 (FIG. 2) may determine the number of anchor points on either side of a moved anchor point which may be changed, adjusted, modified, etc. The processor circuit 210 may adjust the number of anchor points allowed to be changed, adjusted, or modified, in response to a user input or automatically (e.g. based on the field of view of different intraluminal imaging devices used, the zoom setting of the user of the system 100, or any other suitable parameters).

As shown, to generate the contour, the processor circuit 210 (FIG. 2) may be configured to perform interpolation between the plurality of anchors as described with reference to FIG. 4. In addition, the processor circuit 210 (FIG. 2) may be configured to receive a user input to move a location of an anchor (e.g. anchor 1136 of FIG. 11 or any other anchor), and the processor circuit 210 may be configured to perform re-interpolation between only a subset of the plurality of anchors proximate to the anchor (e.g. the anchors 1124, 1135, 1137, and 1138 of FIG. 11). The anchors proximate the moved anchor 1136 may include any number of anchors on either side of the moved anchor 1136 including one, two, three, or more. These anchors may be next to, consecutive with or consecutive to, or adjacent to the anchor 1136. They may be along the contour. These anchors may additionally be immediately following or next to one another along the contour.

FIG. 13 is a diagrammatic view of a graphical user interface 1300 displaying an interpolated contour 1330 with automatically placed anchors 1332 identifying the walls of a vessel into which the intravascular imaging device is not inserted, according to aspects of the present disclosure. The graphical user interface 1300 may be substantially similar to the interface 300 of FIG. 3 and FIG. 4. The interface 1300 may be displayed to the user of the system 100 during or after an imaging procedure. The graphical user interface includes an IVUS image 1320 and an ILD 1340.

The graphical user interface 1300 displays a contour 1330 overlaid on the IVUS image 1320. The contour 1330 may identify the inner walls of a peripheral blood vessel 1392, however, as shown in FIG. 13, the contour 1330 may identify the walls of a vessel that does not contain the intravascular imaging device 102. The imaging device 102 may be seen in the IVUS image 1320 at the center of the image 1320 and within a different vessel than the one identified by the contour 1330. Indeed, an advantage of the algorithm disclosed herein may include the ability to generate contours, such as 1330 around structures, such as neighboring vessels, that do not include the intravascular imaging device 102. For example, the algorithm may allow a contour to be generated on any part of an IVUS image whether or not it includes the center of the image. In addition, as previously stated, the processor circuit may generate contours that can cross a single scan line multiple times. The inner region enclosed by the contour 1330 may represent a cross-sectional shape or area through which blood may flow within the neighboring vessel. The contour 1330 may be generated in response to a user of the system 100 identifying an IVUS image 1320 of interest. The contour 1330 may be generated by the processor circuit of the system 100 in response to various user inputs including any of those previously described. In this way, the processor circuit 210 (FIG. 2) may be configured to generate a contour correspond to an anatomical boundary by connected the plurality of anchors, wherein the contour does not surround a position of the intravascular imaging catheter in the IVUS image.

In some embodiments, the processor circuit 210 (FIG. 2) may be configured to identify a contour (e.g., the contour 1330) of any anatomical structure shown within the image 1320. For example, the anatomical boundary can be a boundary of the body lumen itself, or a body lumen in which an imaging device is positioned (e.g., a vessel lumen boundary, a vessel boundary, such as a boundary of the intima, media, adventitia, external elastic lamina, internal elastic lamina, etc.). In other examples, the anatomical boundary can be a boundary of anatomy different than the body lumen in which the imaging device is positioned, as shown in FIG. 13. In some examples, the imaging device may be a catheter positioned inside an artery while the anatomical boundary identified by the processor circuit may be a boundary of another artery, vein, or other vessel. In some examples, a catheter may be positioned inside vein and the anatomical boundary identified by the processor circuit could be different artery, vein, or other vessel. In some embodiments, the anatomical boundary identified by the processor circuit may be a boundary of any other anatomical structure or man-made structure shown within the image 1320. For example, the boundary may be a boundary of a nerve, ligament, bone, organ, another body lumen, treatment device such as a stent or balloon, or any other structure proximate to the body lumen in which the imaging device is positioned.

Similar to the contours previously discussed, the generation of the contour 1330 may assist the physician or user of the system 100 with performing measurements of aspects of the IVUS image 1320. For example, the user of the system 100 may acquire measurements such as diameters, cross-sectional areas or other measurements of the neighboring vessel identified in FIG. 13. the imaged vessel.

The ILD 1340 may be substantially similar to the ILD 340 described with reference to FIGS. 3 and 4. It may assist a user of the system 100 to identify where along the imaged vessel the IVUS image 1320 was obtained.

After the contour 1330 is generated, the system 100 may perform the steps of smoothing, automatically generating and placing anchors, and interpolating between anchors according to the methods described herein. These steps may include additional steps before, after, or in between these steps. In some embodiments, one or more of these steps may be omitted, performed in a different order, or performed concurrently. These steps can be carried out by any suitable component within the system 100 and all steps need not be carried out by the same component.

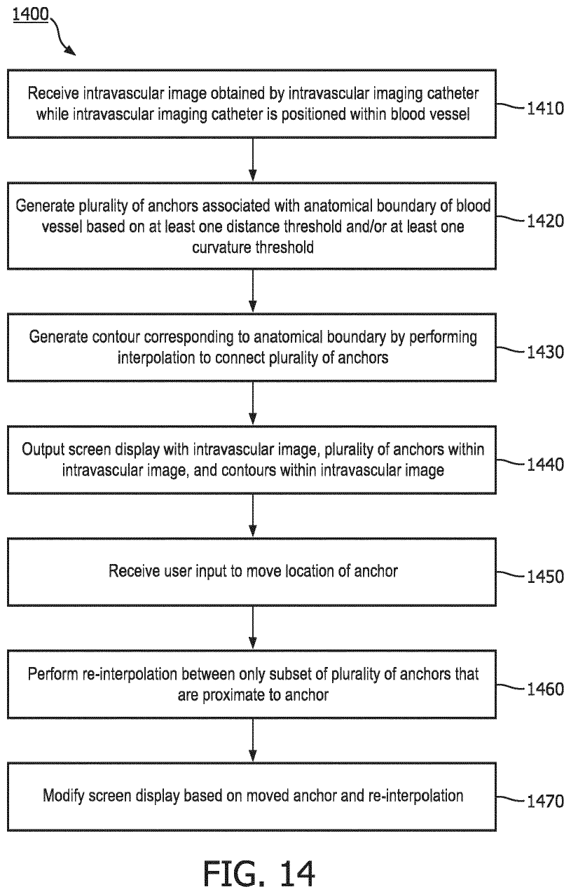

FIG. 14 is a flow diagram for a method 1400. The method 1400 can be referenced as an imaging method, an intravascular imaging method, an ultrasound imaging method, and/or an intravascular ultrasound (IVUS) imaging method in various instances. The method 1400 can be related to generating a contour, automatically generating and placing anchors along the contour, and interpolating between the anchors, according to aspects of the present disclosure. As illustrated, the method 1400 includes a number of enumerated steps, but embodiments of the method 1400 may include additional steps before, after, or in between the enumerated steps. In some embodiments, one or more of the enumerated steps may be omitted, performed in a different order, or performed concurrently. The steps of the method 1400 can be carried out by any suitable component within the system 100 and all steps need not be carried out by the same component. In some embodiments, one or more steps of the methods 1400 can be performed by, or at the direction of, a processor circuit of the system 100 (e.g., the processor circuit 210 of FIG. 2), including, e.g., the processor 260 or any other component.

At step 1410, the method 1400 includes receiving an intravascular image obtained by an intravascular imaging catheter while the intravascular imaging catheter is positioned within a blood vessel. For example, the processor circuit can receive an IVUS image obtained by the IVUS imaging catheter while the IVUS imaging catheter is positioned within a blood vessel.

At step 1420, the method 1400 includes generating a plurality of anchors associated with an anatomical boundary of the blood vessel. The anchor locations are based on at least one distance threshold representative of the distance of the contour between the plurality of anchors and/or at least one curvature threshold representative of the curvature of the contour between the plurality of anchors. For example, the processor circuit can generate a plurality of anchors associated with an anatomical boundary of the blood vessel. The anchor locations are based on at least one distance threshold (e.g., a minimum distance threshold and/or a maximum distance threshold) representative of a distance of the contour between two consecutive anchors and/or at least one curvature threshold (e.g., a maximum negative curvature threshold and/or a maximum positive curvature threshold) representative of a curvature of the contour between the two consecutive anchors.

At step 1430, the method 1400 includes generating a contour corresponding to the anatomical boundary by performing interpolation to connect the plurality of anchors. For example, the processor circuit can generate a contour corresponding to the anatomical boundary by performing interpolation to connect the plurality of anchors.

At step 1440, the method 1400 includes outputting, to a display, a screen display with the intravascular image, the plurality of anchors within intravascular image, and the contour within the intravascular image. For example, the processor circuit can output, to a display in communication with the processor circuit.

At step 1450, the method 1400 includes receive a user input to move the location of an anchor. For example, the processor circuit can receive the user input via a user input device (e.g., touch screen, bedside controller, mobile device, keyboard, mouse, and/or combinations thereof) in communication with the processor circuit.

At step 1460, the method 1400 includes performing re-interpolation between only a subset of the plurality of anchors that are proximate to the moved anchor. For example, the processor circuit can perform re-interpolation to re-generate only a portion of contour within one, two, three, four, five, or more anchors on either side or both sides of the moved anchor, while the other portions of the contour remain the same.

At step 1470, the method 1400 includes modifying the screen display based on the moved anchor and the re-interpolation. For example, the processor circuit can output, to the display, the intravascular image, the plurality of anchors (include the moved anchor at the modified location) within intravascular image, and the contour within the intravascular image. For example, the processor circuit can output, to the display, a screen display comprising: the IVUS image, the plurality of anchors within the IVUS image, and the contour (partially re-interpolated, e.g., only re-interpolated in the region proximate to the moved anchor) within the IVUS image.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an intraluminal imaging catheter and a display, wherein the processor circuit is configured to:
receive an intraluminal image obtained by the intraluminal imaging catheter while the intraluminal imaging catheter is positioned within a body lumen;
generate a plurality of points associated with an anatomical boundary;
generate a contour corresponding to the anatomical boundary by connecting the plurality of points; and
output, to the display, a screen display comprising the intraluminal image, the plurality of points within the intraluminal image, and the contour within the intraluminal image,
wherein, to generate the plurality of points, the processor circuit is configured to determine a plurality of locations for the plurality of points based on:
at least one distance threshold representative of a distance of the contour between the plurality of points; and
at least one curvature threshold representative of a curvature of the contour between the plurality of points.

2. The system of claim 1,
wherein the processor circuit is configured to determine a location of a second point based on at least one of the distance or the curvature of the contour between a first point and the second point, and
wherein the first point and the second point are consecutive.

3. The system of claim 2,
wherein the at least one distance threshold comprises a minimum distance threshold, and
wherein, to determine the location of the second point, the processor circuit is configured to place the second point only when the distance of the contour between the first point and the second point is equal to or greater in magnitude than the minimum threshold distance.

4. The system of claim 3, wherein the processor circuit is configured to change the minimum threshold distance.

5. The system of claim 2,
wherein the at least one distance threshold comprises a maximum distance threshold, and
wherein, to determine the location of the second point, the processor circuit is configured to always place the second point when the distance of the contour between the first point and the second point is equal to or greater in magnitude than the maximum threshold distance.

6. The system of claim 3, wherein the processor circuit is configured to change the maximum threshold distance.

7. The system of claim 2,
wherein the at least one curvature threshold comprises a maximum negative curvature threshold, and
wherein, to determine the location of the second point, the processor circuit is configured to place the second point when the curvature of the contour between the first point and the second point is equal to or less in magnitude than the maximum negative curvature threshold.

8. The system of claim 7,
wherein the processor circuit is configured to place the second point only when the distance of the contour between the first point and the second point is equal to or greater in magnitude than the minimum threshold distance.

9. The system of claim 2,
wherein the at least one curvature threshold comprises a maximum positive curvature threshold, and
wherein, to determine the location of the second point, the processor circuit is configured to place the second point when the curvature of the contour between the first point and the second point is equal to or greater in magnitude than the maximum positive curvature threshold.

10. The system of claim 9,
wherein the processor circuit is configured to place the second point only when the distance of the contour between the first point and the second point is equal to or greater in magnitude than the minimum threshold distance.

11. The system of claim 2,
wherein, to determine the location of the second point, the processor circuit is configured to place the second point when the curvature of the contour between the first point and the second point changes from positive to negative or from negative to positive.

12. The system of claim 11,
wherein the processor circuit is configured to place the second point only when the distance of the contour between the first point and the second point is equal to or greater in magnitude than the minimum threshold distance.

13. The system of claim 1, wherein, to generate the contour, the processor circuit is configured to perform modified Akima piecewise cubic Hermite interpolation between the plurality of point s.

14. The system of claim 1,
wherein, to generate the contour, the processor circuit is configured to perform interpolation between the plurality of point s,
wherein the processor circuit is configured to receive a user input to move a location of a point,
wherein the processor circuit is configured to perform re-interpolation between only a subset of the plurality of point s that is proximate to the point.

15. The system of claim 1, wherein the contour does not surround a position of the intraluminal imaging catheter in the intraluminal image.

16. The system of claim 1, wherein the processor circuit is configured generate the plurality of points automatically without receiving a user input identifying a plurality of locations for the plurality of points.

17. The system of claim 1, further comprising the intraluminal imaging catheter.

18. A system, comprising:
a processor circuit configured for communication with an intraluminal imaging catheter and a display, wherein the processor circuit is configured to:
receive an intraluminal image obtained by the intraluminal imaging catheter while the intraluminal imaging catheter is positioned within a body lumen;
automatically generate a plurality of points associated with an anatomical boundary without receiving a user input identifying a plurality of locations for the plurality of points, wherein the plurality of points include a first point, a second point, and a third point;
generate a contour corresponding to the anatomical boundary by connecting the plurality of points; and
output, to the display, a screen display comprising the intraluminal image, the plurality of points within the intraluminal image, and the contour within the intraluminal image,
wherein, to generate the plurality of points, the processor circuit is configured to determine the plurality of locations for the plurality of points such that a first distance of the contour between the first point and the second point is different than a second distance of the contour between the second point and the third point.

19. A system, comprising:
a processor circuit configured for communication with an intraluminal imaging catheter and a display, wherein the processor circuit is configured to:
receive an intraluminal image obtained by the intraluminal imaging catheter while the intraluminal imaging catheter is positioned within a body lumen;
generate a plurality of points associated with an anatomical boundary;
generate a contour corresponding to the anatomical boundary by connecting the plurality of points; and
output, to the display, a screen display comprising the intraluminal image, the plurality of points within the intraluminal image, and the contour within the intraluminal image,
wherein, to generate the plurality of points, the processor circuit is configured to determine a plurality of locations for the plurality of points based on a change in a curvature of the contour between the plurality of points.
